(19) United States
(12) Patent Application Publication
(10) Pub. No.: US 2005/0080376 A1
(43) Pub. Date: Apr. 14, 2005

(54) Tool for installing a breast interface cup on a breast pump

(76) Inventors: Kenneth Myers, Marietta, CA (US); Drew Meincke, Woodstock, GA (US)

Correspondence Address: MEHRMAN LAW OFFICE, P.C., ONE PREMIER PLAZA, 5605 GLENRIDGE DRIVE, STE. 795, ATLANTA, GA, 30342, US

(21) Appl. No.: 10/683,748

(22) Filed: Oct. 10, 2003

(51) Int. Cl. A61M 1/06

(52) U.S. Cl. 604/74

(57) ABSTRACT

A manually operated breast cup insertion tool for installing the breast interface cup in the breast pump. The tool includes a seating device that is configured to press a seal on the breast interface cup into a seat on the breast pump when the breast interface cup is received on the breast pump, the tool is placed against the breast interface cup, and the tool is pressed toward the breast pump to seat the breast interface cup on the breast pump. For example, the seating device may be a ridge used to press the seal on the breast interface cup into the seat on the breast pump. The tool may also include a guide configured to align the seating device with the seal when the guide is received within a channel of the breast interface cup.

### TECHNICAL FIELD

The present invention relates to breast pumps for expressing breast milk from lactating mothers and, more particularly, relates to a tool for installing a breast interface cup on a breast pump.

### BACKGROUND OF THE INVENTION

Breast pumps for lactating mothers have been commercially available for many years. A major advance in breast pump technology occurred with the advent of a hands-free breast pump held between the mother's breast and her brazier. This type of breast pump includes a soft breast interface cup that fits within the pump and, when worn properly, forms an air-tight seal with the mother's breast. This allows the breast interface cup to function as a suction cup that applies a pumping action to the mother's breast to induce the expression of breast milk.

For the pump to operate properly, the breast interface cup should be properly seated within the breast pump to ensure that an air-tight seal is maintained from the mother's breast to the milk collection bag. However, some users have difficulty properly seating the breast interface cup in the breast pump, which can lead to less than desired results when using the pump. Accordingly, there is an need for a simple, cost effective and substantially error proof way to properly seat the breast interface cup in the breast pump.

### SUMMARY OF THE INVENTION

The present invention meets the needs described above in a manually operated breast cup insertion tool for installing the breast interface cup in the breast pump. The tool includes a seating device that is configured to press a seal on the breast interface cup into a seat on the breast pump when the breast interface cup is received on the breast pump, the tool is placed against the breast interface cup, and the tool is pressed toward the breast pump to seat the breast interface cup on the breast pump. For example, the seating device may be a ridge used to press the seal on the breast interface cup into the seat on the breast pump.

The breast cup insertion tool typically includes a platform that larger than the opening of a channel in a breast interface cup and a guide sized to be received within the cup channel. More specifically, the guide aligns the plunger matches the configuration of the channel within the breast interface cup sufficiently to allow the cup itself to be properly seated in the channel by pressing the tool into the cup while the cup is located within the pump. The tool also includes a pressure relief device that vents the breast cup channel as the plunger is moved within the channel. The plunger may also include a seating device, such as ridge that corresponds to a ledge in the cup channel to help seat the cup against a seal seat of the breast pump.

The breast cup insertion tool allows the user of the breast pump to easily press the breast interface cup into a proper seat in the breast pump. The same breast cup insertion tool can, obviously, be used over and over again to seat breast interface cups into breast pumps. Therefore, the breast cup insertion tool may be sold as a separate item or it may be provided as part of an assembly along with the breast pump and one or more breast interface cups. Accordingly, it should be understood that the present invention may be practiced as a breast cup insertion tool, or as a breast pump assembly including a breast cup insertion tool, a breast pump and a breast interface cup. The invention may also be practiced as a method for installing a breast interface cup in a breast pump using a breast cup insertion tool.

Generally described, the invention may be realized in a tool for installing a breast interface cup on a breast pump. The tool includes a seating device configured to press a seal on the breast interface cup into a seat on the breast pump when the breast interface cup is received on the breast pump, the tool is placed against the breast interface cup, and the tool is pressed toward the breast pump to seat the breast interface cup on the breast pump. The tool may also include a guide configured to align the seating device with the seal when the guide is received within a channel of the breast interface cup.

The seating device typically includes some type of a ridge that is used to press the seal on the breast interface cup into the seat on the pump. Fur example, the breast cup insertion tool may include shunt attached to the guide, and the seating device may include a ridge on the shunt. To make the tool easy to handle, it may also include a platform attached to the shunt.

More specifically described, the platform may include a substantially flat disk having a radius defined about a central axis. In addition, the guide may include a substantially cylindrical outer surface that is coaxial with the platform, extends away from the platform in the axial direction between a proximal end and a distal end, and has a radius that is smaller than the radius of the platform. And the shunt may be located axially between the platform and the proximal end of the guide. The shut may also include a substantially cylindrical outer surface that is coaxial with the platform and the guide, extends between the platform and the proximal end of the guide, and has a radius that is smaller than the radius of the platform and larger than the radius of the guide.

For this configuration, the seating device may include a ridge along the outer surface at the distal end of the shunt. This particular tool configuration may also include a pressure relief device defined by the guide for venting the channel as the guide moves within the channel. For example, the pressure relief device may include an axial slot in the outer surface of the guide, and may further include a notch through the ridge aligned with the slot. In addition, this embodiment of the breast insertion tool may include a radial surface at the distal end of the guide and a beveled edge between the radial surface and the guide outer surface.

Stated somewhat differently, the invention may be practiced as a tool for installing a breast interface cup in a breast pump. This tool may include a platform and a shunt extending in a longitudinal direction from the platform including a proximal end attached to the platform and a distal end. The shunt may also include a seating device configured to press a seal on the breast interface cup into a seat on the breast pump. The breast cup insertion tool may also include a guide extending from the distal end of the shunt in the longitudinal direction. This guide may include a proximal end attached to the distal end of the shunt, and the guide may also be configured to align the seating device with the seal when the guide is received within a channel of the breast interface cup. For this embodiment, the seating device may include a ridge along the distal end of the shunt, and the platform, shunt and guide may have successively narrower transverse dimensions. This embodiment may also include a pressure relief device defined by the guide for venting the channel as the guide moves within the channel.

The invention may also be practiced as a breast pump assembly that includes a breast pump, a breast interface cup having a channel configured to be received within the breast pump, and a tool for installing the breast interface cup on the breast pump. As noted previously, this tool includes a seating device configured to press a seal on the breast interface cup into a seat on the breast pump when the breast interface cup is received on the breast pump, the tool is placed against the breast interface cup, and the tool is pressed toward the breast pump to seat the breast interface cup on the breast pump. The tool may also include a guide configured to align the seating device with the seal when the guide is received within a channel of the breast interface cup.

The invention may also be practiced as a method for installing a breast interface cup on a breast pump. A tool is provided for installing the breast interface cup in the breast pump that includes a seating device configured to press a seal on the breast interface cup into a seat on the breast pump when the breast interface cup is placed into the breast pump, the tool is placed adjacent against the breast interface cup, and the tool is pressed toward the breast pump to seat the breast interface cup on the breast pump. The breast interface cup is placed on the breast pump, the tool is placed against the breast interface cup, and the tool is pressed toward the breast pump to seat the breast interface cup on the breast pump.

In view of the foregoing, it will be appreciated that the present invention provides a simple, cost effective and substantially error proof way to properly seat a breast interface cup on a breast pump. The specific techniques and structures for implementing the breast cup insertion tool, and thereby accomplishing the advantages described above, will become apparent from the following detailed description of the embodiments and the appended drawings and claims.

### BRIEF DESCRIPTION OF THE DRAWINGS

FIG. 1 is a perspective view of a breast cup insertion tool.

FIG. 2 is a crosssection side view of the breast cup insertion tool.

FIG. 3 is an end view of the breast cup insertion tool.

FIG. 4 is a side view of a breast pump assembly including a breast pump, a breast interface cup, and a breast cup insertion tool.

FIG. 5 is a side view of the breast pump assembly with the breast interface cup located within the breast pump.

FIG. 6 is a side view of the breast pump assembly with the breast cup insertion tool located within the breast interface cup while the cup is located within the pump.

FIG. 7 is a side view of the breast interface cup showing the location of the seal on the outside of the cup channel.

FIG. 8 is a perspective view of the breast pump showing the location of the seat within the pump channel for receiving the seal n the outside of the cup channel.

### DETAILED DESCRIPTION OF THE EMBODIMENTS

The present invention may be embodied in a breast cup insertion tool that is used to seat a breast cup in a breast pump. The particular breast cup insertion tool described below is configured to work with the specific breast pump and breast interface cup described in commonly-owned U.S. patent application Ser. No. 10/173,566 filed Jun. 19, 2002 and entitled “Syguide for a Portable Hands-Free Breast Pump and Method for Using the Same,” which is incorporated herein by reference. However, those skilled in the art will understand that the breast cup insertion tool should be configured to match the seal profile of the breast insertion cup that it is configured to seat. As a result, those skilled in the art will understand how to modify the particular breast cup insertion tool described below to work with different breast interface cups and breast pumps. Therefore, the invention in not limited to the particular embodiment described below and shown in the appended figures.

The breast cup insertion tool is preferably injection molded from plastic in two pieces, the platform and the plunger, which are glued together form the insertion tool. However, any suitable material and method of forming the material into the insertion tool may be employed. In addition, the plunger preferably includes a shunt extending from the platform and a narrower guide extending from the shunt. The guide is configured to align a seating device, such as a ridge on the shunt, with a seal on the breast interface cup when the guide is received within a channel of the cup. In this configuration, the pressure relief device is typically configured as a slot in guide aligned with a notch though a ridge formed around the shunt. Of course, other plunger configurations may be employed, and the pressure relief device may be configured as a device other than a slot aligned with a notch. For example, the pressure relief device may be tube, openings to a hollow body of the breast cup insertion tool, or another or other suitable venting device.

Notwithstanding these alternative embodiments that may be used to practice the invention, it should also be appreciated that the particular embodiment shown in the figures and described below has been engineered to be inexpensive to construct, easy to use, and substantially error proof when used properly. For these reasons, this particular configuration is presently considered to be the most technically and economically sound alternative for implementing the invention for use with the particular breast pump and breast interface cup shown and described in this specification.

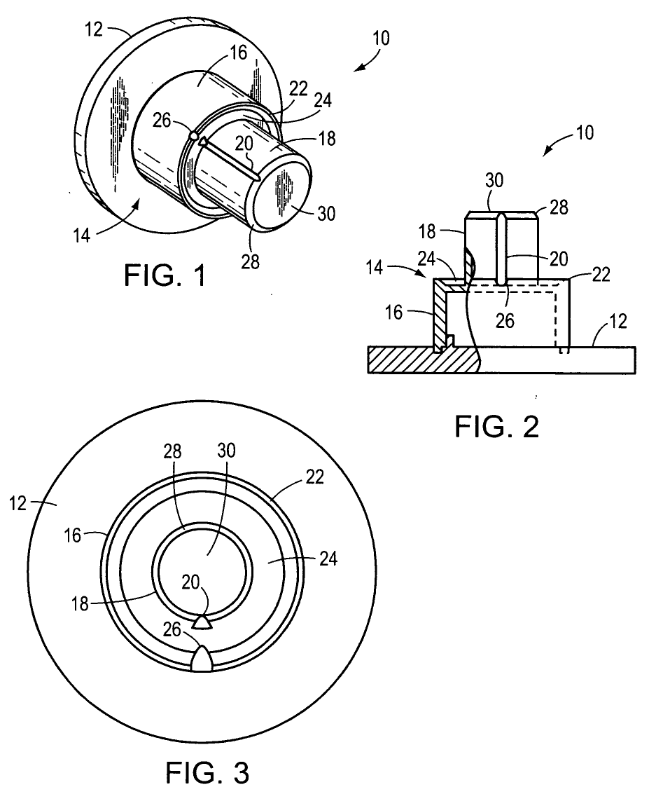

Turning now to the figures, in which like numerals refer to similar elements throughout the several figures, FIG. 1 is a perspective view of a breast cup insertion tool 10. FIG. 2 shows a crosssection side view and FIG. 3 shows an end view of this breast cup insertion tool. The breast cup insertion tool 10 includes disk-shaped platform 12 and a plunger 14 extending from the platform. Since the platform 12 is disk-shaped, it may be described as having a radius defined about a central axis. The platform 12 may also be described as having a transverse dimension (i.e., width) in the radial direction and a longitudinal dimension (i.e., thickness) in the axial direction. Using this nomenclature, the plunger 14 is coaxial with the platform 12 and extends transversely from the platform in the axial direction.

As described more fully below, the plunger 14 includes a shunt 16 and a guide 18 that extends from the shunt. The shunt 16 includes a seating device, in this embodiment a ridge 22 along the outer edge of the of the shunt 16 adjacent to the location where the guide 18 that extends from the shunt. The guide 18 is configured to align the seating device, in this particular case the ridge 22 along the outer edge at the end of the shunt 16, with a seal on the breast interface cup when the guide is received within a channel of the cup.

In addition, the platform 12 may have a transverse dimension that is larger than the opening of the channel to make the breast cup insertion tool easier to handle. However, it should be appreciated that the tool will work properly without the platform 12, which should therefore be considered an optional feature. The guide 16 also conforms to the interior shape of the upper portion of the channel of the breast interface cup near the cup opening sufficiently to be received within the channel. Although the guide 16 need not match the profile of the upper portion of the channel precisely, it should be sufficient close to align the ridge 22 with a seal on the breast interface cup closely enough to aloe the tool 10 to seat the seal in a seat on the breast pump when the tool is presses toward the pump. This, of course, is the purpose of function of the breast insertion tool 10. To accomplish this result, the guide 16 may precisely match the profile of the upper portion channel, or it may match this portion of the channel at one or more key locations sufficient to ensure proper seating of the cup in the pump. Whatever the specific configuration of the guide 16 may be, that configuration should allow the breast cup insertion tool 10 to be used to seat the breast interface cup in the breast pump by pressing the tool toward the pump while the cup is located on the pump.

The particular plunger 14 of the illustrative embodiment shown in FIGS. 1-3 includes a cylindrical shunt 16 having a proximal end attached to the platform 12 and a distal end attached to a cylindrical guide 18. The guide 18, in turn, has a proximal end attached to the distal end of the shunt and a distal end located away from the shunt. In other words, the shunt 16 and the guide 18 extend longitudinally away from the platform 12 in the axial direction. In addition, the platform 12, the shunt 16 and the guide 18 are coaxial and form a series of tiers with reducing radius from the platform to the shunt and then to the guide 18. That is, the radius of the platform 12 is greater than the radius of the shunt 16, which is greater then the radius of the guide 18. It should be appreciated that the use of a “radius” as a defining dimension is appropriate for this particular embodiment because the platform 12, the shunt 16 and the guide 18 are circular in crosssection. For alternative embodiments in which the these components are not circular in crosssection, the platform 12 may be described as having at least one transverse dimension that is greater than at least one transverse dimension of the shunt 16, which is greater than at least one transverse dimension of the guide 18.

The guide 18 also includes a pressure relief device that vents the channel of the breast interface cup as the guide moves within the channel. In this particular embodiment, the pressure relief device is a longitudinal slot 20 along the outer surface of the guide 18. The distal end of the shunt 16 also includes a ridge 22 around the outer surface of the shunt that is configured press a seal in the breast interface cup against a seal seat of the breast pump. This seal seat may extend all the way around the channel of the breast interface cup, or it may be present only at one or more key locations in the channel. For example, the seal seat may formed as a ledge, a channel, a grove, or any other suitable structure or combination of structures. In this regard, many different seal seating arrangement may operate properly, and the particular seal seating arrangement of a particular breast interface cup is a matter of design choice. Whatever the seal seating arrangement of a particular breast cup may be, the plunger 14 of the breast cup insertion tool 10 should be configured to correspond to that seal seating arrangement sufficiently to effect a proper seat of the cup in the pump.

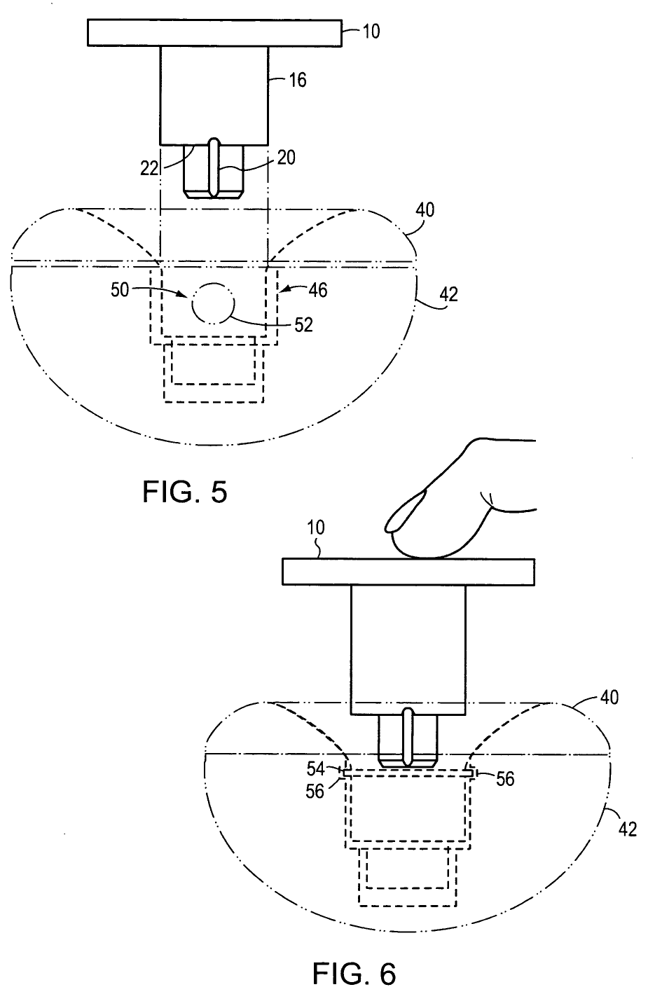

For the particular embodiment shown in FIGS. 1-3, the ridge 22 at the distal end of the shunt 16 functions as a seal seating device, which is aligned with a bump-shaped seal (element 54 shown best on FIG. 5) when the guide 18 is placed in the channel of the breast interface cup. Specifically, the ridge 22 is positioned on the inside of the channel adjacent to and on the opposing side of the seal 54, which his located on the other side of the channel adjacent to the breast pump. In addition, a radial surface 24 extends from the ridge 22 to the junction between the distal end of the shunt 16 and the proximal end of the guide 18. For this configuration, the pressure relief device also includes a notch 26 through the ridge 22 aligned with the slot 20, as shown best in FIG. 1.

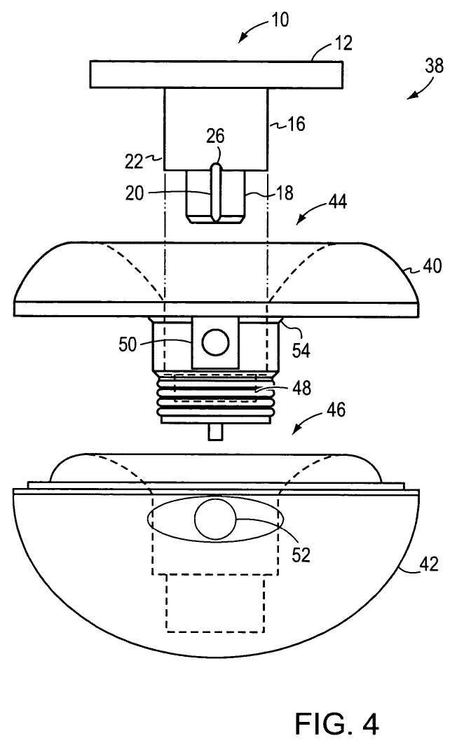

FIG. 4 is a side view of a breast pump assembly 38 including a breast cup insertion tool 10, a breast interface cup 40, and a breast pump 42. The breast interface cup 40 includes a cup channel 44 that fits into a pump channel 46 in the breast pump 42. The guide 18 of the breast cup insertion tool 10 is configured to be received within the cup channel 44, which aligns the ridge 22 in the end of the shunt 16 with the seal 54, which is opposing side of the cup channel 44. In this particular embodiment, the distal end of the guide 18 also includes a beveled lip 28 between the outer surface of the guide and a radial surface 30 located at the distal end of the guide, as shown best in FIG. 1. The beveled lip helps the guide 18 slip easily into the cup channel 44.

FIG. 5 is a side view of the breast pump assembly 38 with the breast interface cup 40 located within the breast pump 42. In this particular breast pump assembly 38, the breast interface cup 40 includes a cup port 50 that is aligned with a pump port 52 when the cup is properly installed within the pump. A one-way valve removably attaches to the cup port 50, and a guide leading to a milk collection bag extends through the pump port 52 and removably attaches to the one-way valve. For this reason, properly seating the breast interface cup 40 in the breast pump 42 should cause the cup port 50 become aligned with a pump port 52. Therefore, the cup port 50 of the breast interface cup 40 should be aligned with the pump port 52 of the pump 42 before the breast cup insertion tool 10 is placed against into the breast cup to seat the cup within the pump. The user then presses the breast cup insertion tool 10 toward the pump 42, as shown in FIG. 6, to seat the breast interface cup 40 in the pump 42. As noted previously, the ridge 22 along the outer surface of the distal end of the shunt 16 presses the seal 54 on the exterior the cup channel 44 into the seat 56 on the breast pump to properly seated the breast interface cup 40 on the pump 42. In this particular configuration, the seal 54 is a bump-shaped projection that extends most of the way around the exterior the cup channel 44, and the seat 56 is a matching groove around the pump channel 46.

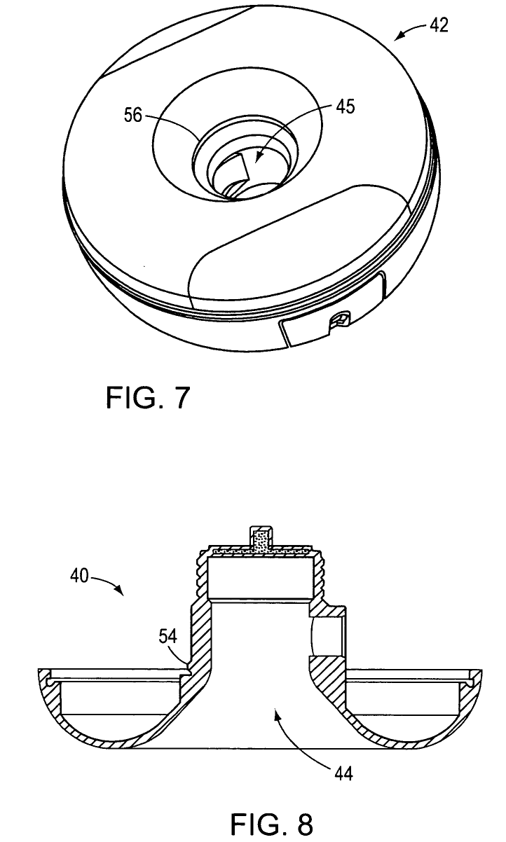

FIG. 7 is a side view of the breast interface cup 40 showing the location of the seal 54 on the outside of the cup channel 44. The bump-shaped seal 54 extends around the cup channel 44 except in the area of the cup port 50. FIG. 8 is a perspective view of the breast pump 42 showing the location of the seat 56 within the pump channel 46 for receiving the seal 54. Again, the groove-shaped seat 56 extends around the cup channel 44 except in the area of the pump port 52. As described previously with reference the FIGS. 1 and 6, the guide 18 of the breast cup insertion tool 10 is configured to be received within the cup channel 44 to align the ridge 22 along the outer surface of the distal end of the shunt 16 with the seal 54. The breast cup insertion tool 10 is then pressed toward the breast pump 42, which causes the ridge 22 to press the seal 54 into the seat 56 to properly seated the breast interface cup 40 on the pump 42.

In view of the foregoing, it will be appreciated that present invention provides a simple, cost effective and substantially error proof way to properly seat the breast interface cup in the breast pump. It should be understood that the foregoing relates only to the exemplary embodiments of the present invention, and that numerous changes may be made therein without departing from the spirit and scope of the invention as defined by the following claims.

1. A tool for installing a breast interface cup on a breast pump comprising a seating device configured to press a seal on the breast interface cup into a seat on the breast pump when the breast interface cup is received on the breast pump, the tool is placed against the breast interface cup, and the tool is pressed toward the breast pump to seat the breast interface cup on the breast pump.

2. The tool of claim 1, further comprising a guide configured to align the seating device with the seal when the guide is received within a channel of the breast interface cup.

3. The tool of claim 1, wherein the seating device comprises a ridge.

4. The tool of claim 2, further comprising a shunt attached to the guide, and wherein the seating device comprises a ridge on the shunt.

5. The tool of claim 4, further comprising a platform attached to the shunt.

6. The tool of claim 6, wherein:
the platform comprises a substantially flat disk having a radius defined about a central axis;
the guide comprises a substantially cylindrical outer surface that is coaxial with the platform, extends away from the platform in the axial direction between a proximal end and a distal end, and has a radius that is smaller than the radius of the platform, and
the shunt is located axially between the platform and the proximal end of the guide, comprises a substantially cylindrical outer surface that is coaxial with the platform and the guide, extends between the platform and the proximal end of the guide, and has a radius that is smaller than the radius of the platform and larger than the radius of the guide.

7. The tool of claim 6, wherein the seating device comprises a ridge along the outer surface at the distal end of the shunt.

8. The tool of claim 7, further comprising a pressure relief device defined by the guide for venting the channel as the guide moves within the channel.

9. The tool of claim 8, wherein the pressure relief device comprises an axial slot in the outer surface of the guide.

10. The tool of claim 9, wherein the pressure relief device further comprises a notch through the ridge aligned with the slot.

11. The tool of claim 10, further comprising a radial surface at the distal end of the guide and a beveled edge between the radial surface and the guide outer surface.

12. A tool for installing a breast interface cup in a breast pump, comprising:
a platform;
a shunt extending in a longitudinal direction from the platform, the shunt having a proximal end attached to the platform and a distal end, the shunt comprising a seating device configured to press a seal on the breast interface cup into a seat on the breast pump; and
a guide extending from the distal end of the shunt in the longitudinal direction, the guide having a proximal end attached to the distal end of the shunt, the guide configured to align the seating device with the seal when the guide is received within the a channel of the breast interface cup.

13. The tool of claim 12, wherein the seating device comprises a ridge along the distal end of the shunt.

14. The tool of claim 13, wherein the platform, shunt and guide have successively narrower transverse dimensions.

15. The tool of claim 14, further comprising a pressure relief device defined by the guide for venting the channel as the guide moves within the channel.

16. A breast pump assembly comprising:
a breast pump;
a breast interface cup having a channel configured to be received within the breast pump; and
a tool for installing the breast interface cup on the breast pump comprising a seating device configured to press a seal on the breast interface cup into a seat on the breast pump when the breast interface cup is received on the breast pump, the tool is placed against the breast interface cup, and the tool is pressed toward the breast pump to seat the breast interface cup on the breast pump.

17. The breast pump assembly of claim 16, wherein the tool further comprises a guide configured to align the seating device with the seal when the guide is received within a channel of the breast interface cup.

18. The breast pump assembly of claim 16, wherein the tool further comprises:
a platform;
a shunt comprising the seating device extending in a longitudinal direction from the platform, the shunt having a proximal end attached to the platform and a distal end; and
a guide extending from the distal end of the shunt in the longitudinal direction, the guide having a proximal end attached to the distal end of the shunt, the guide configured to align the seating device with the seal when the guide is received within the channel.

19. The tool of claim 18, wherein the seating device comprises a ridge along the distal end of the shunt.

20. A method for installing a breast interface cup on a breast pump, comprising the steps of:
providing a tool for installing the breast interface cup on the breast pump comprising a seating device configured to press a seal on the breast interface cup into a seat on the breast pump when the breast interface cup is placed into the breast pump, the tool is placed adjacent against the breast interface cup, and the tool is pressed toward the breast pump to seat the breast interface cup on the breast pump;
placing the breast interface cup on the breast pump;
placing the tool against the breast interface cup; and
pressing the tool toward the breast pump to seat the breast interface cup on the breast pump.
